(19) United States
(12) Patent Application Publication
(10) Pub. No.: US 2016/0146804 A1
(43) Pub. Date: May 26, 2016

(54) DNA-CONJUGATED ANTIBODIES FOR IMPROVED ANTIBODY AFFINITY AND REDUCED ANTIBODY CROSS REACTIVITY

(71) Applicant: THE BOARD OF TRUSTEES OF THE LELAND STANFORD JUNIOR UNIVERSITY, Stanford, CA (US)

(72) Inventors: Ronald W. DAVIS, Palo Alto, CA (US); Nicholas HURLBURT, Davis, CA (US); Jacob M. ZAHN, San Jose, CA (US); Mehdi JAVANMARD, Sunnyvale, CA (US); Janine A. MOK, Palo Alto, CA (US)

(21) Appl. No.: 14/899,472

(22) PCT Filed: Jun. 26, 2014
(86) PCT No.: PCT/US2014/044434

§ 371 Date: Dec. 17, 2015

Related U.S. Application Data
(60) Provisional application No. 61/840,424, filed on Jun. 27, 2013.

(51) Int. Cl. G01N 33/543

(52) U.S. Cl. CPC G01N 33/54393

(57) ABSTRACT

Disclosed are specific binding molecules (e.g. antibodies) that are provided in a set of conjugates, where each conjugate comprises an antibody linked to an oligonucleotide (oligo), such as a DNA oligonucleotide. The antibodies in each set are to the same target antigen. One antibody is preferably immobilized so that it remains in the sample reaction after a wash step. One antibody is used for detection since it will remain in the sample after washing only if there is a specific antibody-target antigen. The oligos in a given set hybridize to each other when both antibodies bind to a target with immuno-specificity. However, if the binding is not immune specific, such as in the case of cross-reactivity, the oligos do not hybridize.

### CROSS-REFERENCE TO RELATED APPLICATIONS

This application claims priority from U.S. Provisional Patent Application No. 61/840,424 filed on Jun. 27, 2013, which is hereby incorporated by reference in its entirety.

### STATEMENT OF GOVERNMENTAL SUPPORT

This invention was made with Government support under contract HG000205 awarded by the National Institutes of Health. The Government has certain rights in this invention.

### REFERENCE TO SEQUENCE LISTING, COMPUTER PROGRAM, OR COMPACT DISK

None.

### BACKGROUND OF THE INVENTION

1. Field of the Invention

The present invention relates to the field of immunoassays.

2. Related Art

Presented below is background information on certain aspects of the present invention as they may relate to technical features referred to in the detailed description, but not necessarily described in detail. That is, individual compositions or methods used in the present invention may be described in greater detail in the publications and patents discussed below, which may provide further guidance to those skilled in the art for making or using certain aspects of the present invention as claimed. The discussion below should not be construed as an admission as to the relevance or the prior art effect of the patents or publications described.

### Specific Patents and Publications

U.S. Patent Publication 20020064779 by Landegren, et al. published May 30, 2002, entitled “Methods and kits for proximity probing” and U.S. Patent publication 20050233351 ('351) by Landegren, published Oct. 20, 2005, entitled “Ultrasensitive immunoassays” show the idea of having two antibodies with attached polynucleotides, where hybridization will take place when the two antibodies are both bound to the antigen. However, the conjugated oligonucleotides may then serve as a template for a nucleic acid amplification reaction, for detection of said macromolecule.

U.S. Pat. No. 6,927,024 discloses a sandwich immuno-PCR is a modification of the conventional ELISA format in which the detecting antibody is labeled with a DNA label.

### BRIEF SUMMARY OF THE INVENTION

The following brief summary is not intended to include all features and aspects of the present invention, nor does it imply that the invention must include all features and aspects discussed in this summary.

The present invention comprises in certain aspects a novel approach for both improving antibody affinity and reducing antibody cross-reactivity, two characteristics that are particularly important for multiplexed protein analysis. In contrast to traditional approaches for improving antibody quality which involve the optimization of epitope affinity, the present invention has the advantage of being universally applicable to any pre-existing antibody. The invention is particularly suited for immunoassays using monoclonal antibodies to detect an antigen in a sample, and may be adapted to a microfluidic wash and read protocol using known buffers and detection molecules, e.g. anti-IgG antibodies carrying various labels.

Referring now to FIG. 1A and 1B, an antibody, e.g. a monoclonal antibody 104 is immobilized and allowed to bind to an antigen (circle). The monoclonal antibody 104 has covalently conjugated thereto an oligo 108. A second antibody 102, having specificity also for the antigen, but possibly to a different epitope, also has an oligo 106 chemically conjugated to it. The oligos do not separate from the antibodies in use.

The present method incorporates oligonucleotide (oligo) hybridization to increase the specificity of antibodies to their target antigen. This requires two antibody-oligo conjugates for antigen detection, where the antibodies may be the same or different, but are both specific to a single target antigen. The oligos are designed to have complementary ends 109 whereby the 3′ and 5′ end regions of oligos (oligonucleotides) can hybridize to each other if, and only if, the antibodies have both formed an antibody-antigen complex. When a sample is incubated with the antibody-oligo conjugate, a sandwich complex is formed between the two modified antibodies and the antigen of interest. If the sandwich complex forms as a result of specific binding of the antibody to the antigen, the two complementary oligo sequences are brought in close physical proximity of one another, thus facilitating their rapid hybridization.

However, if the sandwich complex is formed as a result of cross-reactivity or nonspecific binding of the antibodies 110, 112 to the antigen (FIG. 1B), the oligos conjugated to the antibodies will not be complementary, and hybridization will not occur, allowing the sandwich complex to be readily separated using wash conditions that disrupt epitope binding of the antibodies.

Antibody cross-reactivity results in significant signal interference, a common obstacle that limits multiplexing in antibody microarray technology. Thus, we are developing the use of oligo-conjugated antibodies as a potential solution for cross-reactivity reduction. In our system, a weak reaction between an antibody and its target antigen will not produce in the polynucleotide hybridization necessary for a signal. For our initial tests, we used vascular endothelial growth factor (VEGF) in a simple ELISA. In our setup, monoclonal mouse anti-human VEGF antibodies were immobilized on the surface of the plate, and detection was achieved using polyclonal goat anti-human VEGF and FITC-conjugated anti-goat IgG antibodies. Both of the anti-human VEGF antibodies were conjugated using N-hydroxysuccinimide ester-containing bifunctional linkers to amine modified 30-mer oligos that shared 20 base pairs of complementarity. Although additional optimizations are contemplated both in terms of the conjugation chemistry to minimize the interference of the conjugated oligo with the epitope binding of the antibodies and washing conditions to more stringently break apart sandwich complexes that form from non-specific binding, these antibody-oligo conjugates were observed to maintain functionality (FIG. 1C).

Thus, the present invention comprises, in certain aspects, a method for detecting a target antigen using a first antibody-like molecule (“first antibody”) which captures the antigen by binding to it, and a second antibody-like molecule (“second antibody”) which also binds to the antigen and is used for detection of the target antigen. The method comprises (a) providing said first antibody and said second antibody with polynucleic acid strands having regions that are complementary to each other; (b) forming a complex between a target antigen, said first antibody and said second antibody; (b) incubating the complex under conditions which allow hybridization of the polynucleic acid strands of step (b) to each other, then (c) subjecting the complex to a washing step which removes nonspecifically bound antibody but not specifically bound antibody; and (d) detecting specifically bound antibody using the second antibody.

In certain aspects, the present methods may comprise steps wherein said first antibody is immobilized on a substrate, whereby the washing permits retention of the antibody in the sample. In certain aspects, the present methods may comprise steps wherein a multiple of target antigens are detected, and the method uses different antibody pairs, each having specificity to a different target antigen. In certain aspects, the present methods may comprise steps wherein the second antibody(ies) are detected immunologically, that is, as opposed to detecting the presence of the oligonucleotides. Such detection may be protein-based, e.g. by anti-antibodies. In certain aspects, the present methods may comprise steps wherein the second antibody(ies) are detected in a microfluidic device. In certain aspects, the present methods may comprise steps wherein the polynucleic acid strands are chemically linked to said first antibody and said second antibody by chemically conjugated to amine-modified oligos. In certain aspects, the present methods may comprise steps wherein the polynucleic acid strands comprise a linkage having N-hydroxysuccinimide ester. In certain aspects, the present methods may comprise steps wherein the first antibody and the second antibody are monoclonal antibodies to different epitopes of the same antigen.

In addition, the present invention contemplates reagents and kits for carrying out the present methods. Provided are kits for specific applications, where a predetermined set of antibody pairs are included in kit for certain applications. In certain aspects, the present invention may comprise kit for carrying out a method for detecting a number of different target antigen in a sample, comprising a plurality of antibody pairs, wherein each pair comprises a first antibody and a second antibody wherein the first antibody and the second antibody can concurrently bind to the target antigen for detection of the target antigen, and further wherein the first antibody is linked to a first polynucleic acid strand and a second antibody is linked to a second polynucleic acid strand, having the a first polynucleic acid strand and the second polynucleic acid strand comprise complimentary portions that permit specific hybridization of the a first polynucleic acid strand to the second polynucleic acid strand when the first antibody is in proximity to the second antibody. As will be understood from the following description, the oligos attached to the antibodies are constructed for specific hybridization to each other and not to oligos in other antibody pairs. The kits will typically contain instructions for use with the above described methods.

### BRIEF DESCRIPTION OF THE DRAWINGS

FIGS. 1A and 1B are schematic drawings that show an overview of the strategy to improve antibody performance using oligo-conjugated antibodies. FIG. 1C is a graph that shows a demonstration of functionality of antibody-oligo conjugates. Bar graph showing averaged normalized signals for VEGF ELISA performed using unconjugated antibodies (Un), oligo-conjugated antibody pairs with complementary oligos (Comp), oligo-conjugated antibody pairs with non-complementary oligos (Non). In the experiments where VEGF antigen was present, a + is indicated, where a − indicates no VEGF antigen. Experiments with elution step are indicated with a + sign. Experiments without elution are indicated by a − sign. Fluorescence readout was obtained using Perkin Elmer's Victor 2 plate reader, and conditions were tested in triplicate.

FIGS. 2A and 2B are graphs that show performance of the present assay. A “Dandybody” (DNA-linked antibody) assay (Db) compared to normal unconjugated antibody ELISA (Ab). The addition of anti-canine VEGF antibodies (ca) results in an increase in signal resulting from spurious cross reactive binding as compared to a normal unconjugated antibody ELISA performed only with anti-human VEGF antibodies (h). Conjugating the anti-human VEGF antibodies with complementary oligos results in reduced cross reactivity. An elution step (elute) disrupts antibody-epitope interactions, stringently washing cross reactive antibodies away. Non-cross reactive antibodies remained bound to the surface through hybridization of the complementary oligos. That is, the oligo on one antibody hybridizes to an oligo on the other antibody. FIG. 2B is the same as 2A, but in a bar format so that the legends are more clear. The legend matches the 8 bars in each set from top to bottom.

FIG. 3 is a schematic showing a monoclonal antibody mAB1 immobilized by its Fc portion and conjugated to an antigen TA, to which a second monoclonal antibody mAB2 also binds, as in FIG. 1. In this embodiment, the oligonucleotides D1 and D2, conjugated to the monoclonals do not directly hybridize. Instead, a third oligo B1 is used to bridge D1 and D2 by hybridizing to both D1 and D2 in different portions of B1 so that it may simultaneously bind D1 and D2.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. As a non-limiting example, a range of 120 to 250 is intended to include a range of 120-121, 120-130, 200-225, 121-250 etc. The term “about” has its ordinary meaning of approximately and may be determined in context by experimental variability. In case of doubt, “about” means plus or minus 5% of a stated numerical value.

The term “antibody” or “antibody like molecule” refers to an antibody, antibody peptide(s) or immunoglobulin, or an antigen binding fragment of any of the foregoing, e.g., of an antibody. The term “antibody” as used herein may refer to a variety of immunologically specific proteins. Antibody molecules useful in the present invention include single chain antibody molecules, e.g., scFv, see. e.g., Bird et al. (1988) Science 242:423-426; and Huston et al. (1988) Proc. Natl. Acad. Sci. USA 85:5879-5883), and single domain antibody molecules, see, e.g., WO9404678. Although not within the term “antibody molecules,” the invention also includes “antibody analog(s),” other non-antibody molecule protein-based scaffolds, e.g., engineered binding proteins, fusion proteins and/or immunoconjugates that use CDRs to provide specific antigen binding. The term “antibody” also includes synthetic and genetically engineered variants.

The antibody(ies) used in the present method may be detected immunologically. That is, the presence of an antibody in the sample by be detected by an anti-antibody, such as an anti-IgG antibody labeled as may be found in indirect ELISAs, e.g. horseradish peroxidase (HRP) and alkaline phosphatase (AP). Other enzymes may be used as well. These include β-galactosidase, acetylcholinesterase and catalase. A large selection of substrates is available for performing the ELISA with an HRP or AP conjugate. The choice of substrate depends upon the required assay sensitivity and the instrumentation available for signal-detection (spectrophotometer, fluorometer or luminometer).

The term “oligonucleotide” or “polynucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alphanumeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoögsteen types of tee pairing, or the like.

The oligos conjugated to the present antibodies or binding molecules are generally in the range of about 10 to 100 bases in length, although the size may vary.

As used herein, the term “hybridize” or “specifically hybridize” refers to a process where two complementary single-stranded nucleic acid strands anneal to each other under appropriately stringent conditions. Hybridizations to target nucleic acids are typically and preferably conducted with probe-length nucleic acid molecules, preferably 20-100 nucleotides in length. Nucleic acid hybridization techniques are well known in the art. See, e.g., Sambrook, et al., Molecular Cloning: A Laboratory Manual, Second Edition, Cold Spring Harbor Press, Plainview, N.Y. (1989); Ausubel, F. M. et al., Current Protocols in Molecular Biology, John Wiley & Sons, Secaucus, N.J. (1994).

An antibody-like molecule is deemed to be specific for an antigen if the EC50 (half maximal effective concentration (EC50)) of the antigen binding moiety is determined to be less than 100 nM, less than 90 nM, less than 80 nM, less than 70 nM, less than 60 nM, less than 50 nM, less than 40 nM, less than 30 nM, less than 29 nM, less than 28 nMm, less than 27 nM, less than 26 nM, less than 25 nM, less than 24 nM, less than 23 nM, less than 22 nM, less than 21 nM, less than 20 nM, less than 15 nM, less than 10 nM, less than 5 nM, less than 4 nM, less than 3 nM, less than 2 nM, less than 1 nM for each of the respective antigens. EC50 can be determined by known methods. The term “affinity” as used herein refers to the strength of interaction between an antigen binding moiety, like e.g. a monoclonal antibody and an antigen at single antigenic sites. Within each antigenic site, the variable region of the antibody “arm” interacts through weak non-covalent forces with antigen at numerous sites; the more interactions, the stronger the affinity. Accordingly non-specific binding is defined as a weak binding that may be determined as defined above as an EC50 more than 100 nM; it may also be determined by Scatchard plot as described in Mendel et al., “Non-specific' binding. The problem, and a solution,” Biochem J. May 15, 1985; 228(1): 269-272 where a concave curve would be observed.

The phrase “cross-reactively binds” and the terms “cross-specific” and “cross-reactive” are used herein interchangeably and refer to an antigen binding region which has the ability to specifically bind to more than one antigen. For example, in the present disclosure an animal antigen may bind cross-reactively to an anti-human protein antibody. Antigen specificity is defined above and can be determined by methods known to one of skill in the art like e.g. ELISA, FACS, Western Blot, Immuno Blot, BlAcore or SET. In the present disclosure an antigen binding moiety is deemed to be cross-reactive to an unlimited number of antigens if the antigen binding moiety is demonstrated to be able to bind to a specific selection of at least two antigens at least 2-fold, at least 3-fold, at least 4-fold, at least 5-fold, at least 6-fold, at least 7- fold, at least 8-fold, at least 9-fold, at least 10-fold, at least 20-fold, over background. Thereby the background is determined by an antigen binding moiety which is known to be unspecific for the selected antigens or by comparison to binding to an unrelated antigen.

### General Method, Materials and Apparatus

The present technique works by conjugating primary and secondary antibodies (both are specific to the target protein) with complementary nucleic acid sequences. By introducing a target protein into the sample, a sandwich complex will form between the primary antibody, the target protein, and the secondary antibody, thus bringing the two DNA strands in proximity with each other. Once the two nucleic acid strands are in proximity with each other, they can hybridize with each other more rapidly. If the sandwich complex between the primary and secondary antibody forms due to cross reactivity between the antibodies and a non-target protein, the two DNA strands will not hybridize with each other.

### Elution of the Antigen-Antibody Interaction

After the complex has formed, a washing buffer can be introduced which will elute the antigen-antibody interaction, while keeping the interactions between the DNA strands intact. As a result, if the formation of the sandwich complex is due to nonspecific binding of the non target antigen and the primary and secondary antibodies, the secondary antibody will be washed off altogether, since the DNA strands have not hybridized with each other to form an extra specific link. In contrast, in the case where the antigen-antibody complex is specific, and thus the complementary DNA strands hybridizing with each other will have formed a second link, the elution of the antigen antibody interaction will not cause the secondary antibody to be washed off from the surface.

### Microfluidic Embodiments

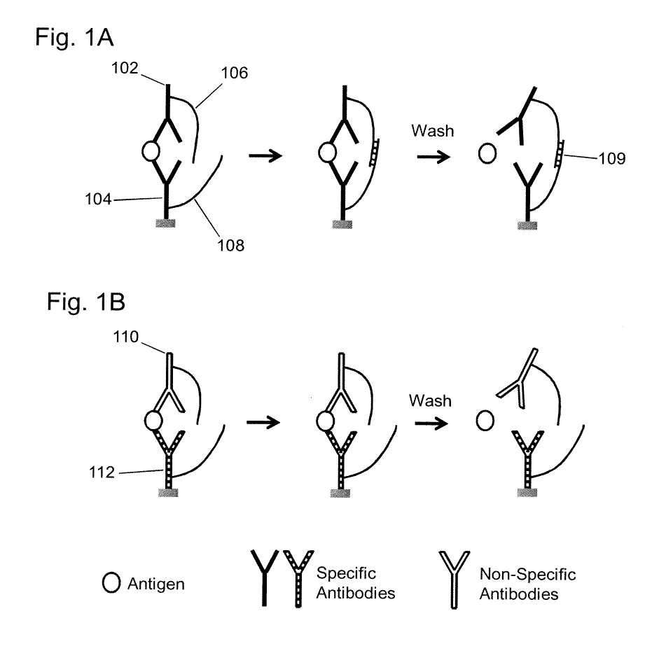

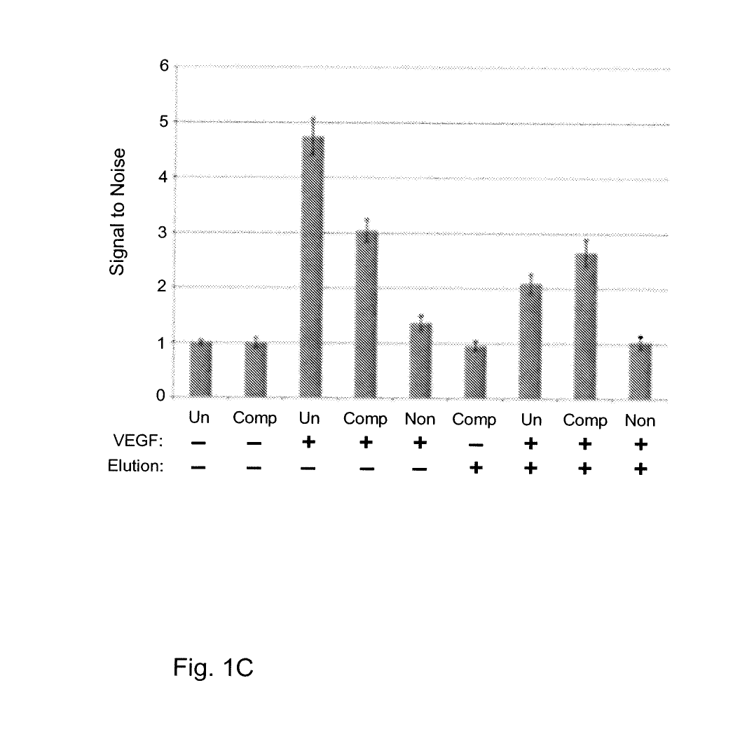

The present methods contemplate detection of the antibody that remains bound after washing (see antibody 102 in FIG. 1A). This antibody may itself be labeled or it may be detected with a secondary antibody, e.g. anti-IgG.

Microfluidics may be used for immobilization of the first antibody (104 in FIG. 1) and/or for washing non-specifically bound second antibody (110 in FIG. 1B). Microfluidics may also be used for detection of the specific complex. This is described in detail in Davis et al. U.S.20100075340 A1, “Electrical Detection Of Biomarkers Using Bioactivated Microfluidic Channels,” which, according to the conclusion of the present specification, is also incorporated herein by reference. The devices of that disclosure can be used to detect and quantify the present complexes by measuring instantaneous changes in ionic impedance. The micro-channel devices of that disclosure are also suitable for the detection of target protein and oligonucleotide, and small molecule target biomarkers using protein-functionalized micro-channels for the rapid electrical detection and quantification of any type of target protein biomarker in a sample. The biochip microfluidic devices may be combined with an integrated circuitry into a portable handheld device for multiplex high throughput analysis using an array of micro-channels for probing clinically relevant samples, such as the human serum, for multiple protein and nucleic acid biomarkers for disease diagnosis, and the detection of potentially pathogenic organisms.

As described above, the present invention utilizes a plurality of binding proteins (e.g. antibodies) that are each separately conjugated to an oligonucleotide (“oligo”) so that when the binding proteins both bind to a target, the oligos can form a hybridization bond through base pairing. This series of Watson-Crick base pair bonds increases antibody specificity and reduces the detection of non-specific binding.

Detection is achieved in three steps: a step that allows the detector antibody to bind in conditions that promote antibody-antigen binding, a step that allows the complementary oligos to hybridize in conditions that promote nucleic acid hybridization, and an elution step in which antibody-antigen interactions are disrupted as to wash away nonspecifically bound antibody-oligo conjugates. When performed in multi-well plate format, the antibody-antigen binding step is one hour long. When performed in multi-well plate format, the nucleic acid hybridization step must be at least 16 hours long; results from shorter incubations suggest a lack of hybridization between the oligos.

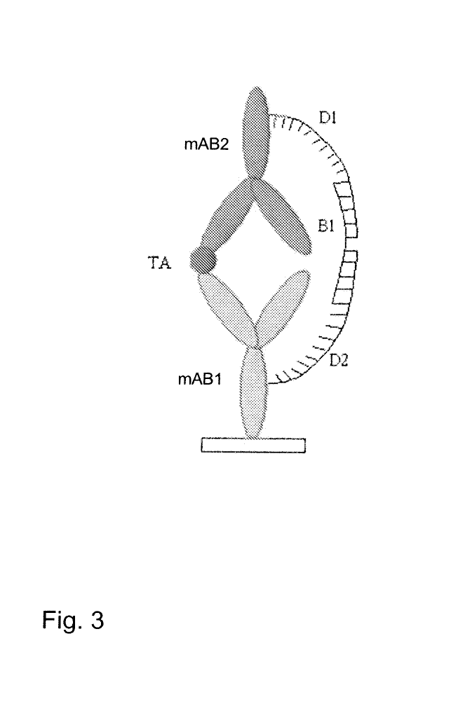

In an alternative embodiment, the oligos may be designed to hybridize to a third, bridge oligo as shown in FIG. 3.

### EXAMPLES

### Example 1: VEGF Detection

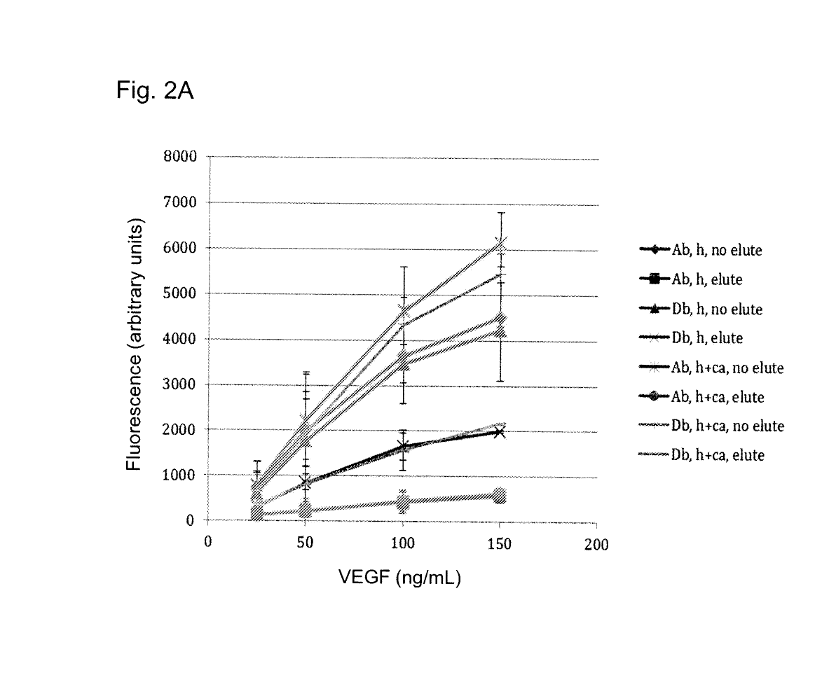

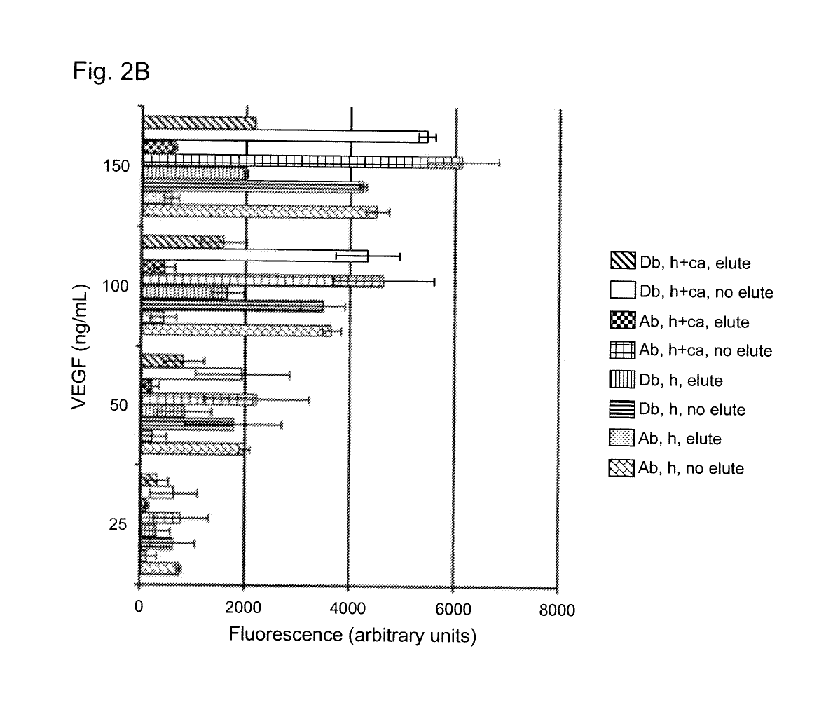

We have performed optimizations of the antibody-oligo conjugation and washing conditions. We used an antibody raised against canine VEGF as a mimic for a cross-reactive antibody that would be present in protein microarrays. Inclusion of this anti-canine VEGF antibody in our human VEGF ELISA results in a false inflation of signal. To demonstrate that our oligo-conjugated antibodies increases specificity, we conjugated both of the anti-human VEGF antibodies and the anti-canine VEGF antibodies to oligos (FIG. 2). The polyclonal anti-human VEGF antibody and the surface-immobilized monoclonal anti-human VEGF antibody were both conjugated to oligos that were complementary in sequence to one another. The polyclonal anti-canine VEGF antibody was conjugated to a noncomplementary oligo. We observed that when human VEGF is present, hybridization of the complementary oligos present on each of the anti-human VEGF antibodies stabilizes the binding of the polyclonal antibody. However, when human VEGF is present and nonspecifically bound by the anti-canine VEGF polyclonal antibody, oligo hybridization does not occur and the sandwich complex is readily separated by washing with an antibody- antigen elution buffer. Thus, the inflated signal observed when both unconjugated polyclonal antibodies are pooled together is reduced with the use of our oligo-conjugated antibodies.

### Example 2: Antibody Conjugation

In terms of the conjugation, we minimized the interference of the conjugated oligo with the epitope binding of the antibodies. Our initial conjugates have been made using N-hydroxysuccinimide ester-containing bifunctional linkers to covalently bond amine-modified oligos 30 bp in length with a 20 bp hybridization region to lysine residues present on the antibody. It was necessary to vary the molar ratio of activated oligos to antibody during the conjugation reaction as to optimize the number of oligos conjugated on each antibody molecule (as specified in the claims). Too many oligos conjugated to the antibody made the oligo-antibody conjugate non-functional. We hypothesize that the abundance of oligos rendered the overall electrostatic charge of the antibody excessively negative, which in turn adversely affected the ability of antigen to recognize its epitope.

We conclude that the antibody-oligo conjugation reaction must be titrated such that less than four oligos are conjugated to each antibody; too many oligos conjugated to each antibody appears to interfere with functionality of the antibody in being able to recognize its target epitope.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

What is claimed is:

1. A method for detecting a target antigen in a sample, using a first antibody and a second antibody which can concurrently bind to the target antigen, comprising:
(a) providing a first antibody linked to a first polynucleic acid strand and a second antibody linked to a second polynucleic acid strand, wherein the first polynucleic acid strand and the second polynucleic acid strand comprise complementary portions that permit hybridization and formation of a polynucleotide complex between the first polynucleic acid strand and the second polynucleic acid strand when the first antibody is in proximity to the second antibody as a result of binding to the target antigen;
(b) forming, in the sample, a complex wherein a target antigen may be bound to said first antibody and also bound to said second antibody;
(c) incubating the complex of step (b) under conditions which allow polynucleic acid strands of step (a) to hybridize and form a polynucleotide complex between the first polynucleic acid strand and the second polynucleic acid strand;
(d) subjecting the complex in step (c) to a wash step which removes any nonspecifically bound antibody from the target antigen but does not remove any specifically bound antibody, whereby polynucleotide hybridization occurs; and
(e) detecting a presence in the sample of an antibody after the wash of step (d), wherein antibody not having a hybridizing strand is removed in step (d) and wherein presence of the antibody in step (e) having a hybridizing strand indicates specific binding to the target antigen.

2. The method of claim 1 wherein said first antibody is a monoclonal antibody immobilized on a substrate.

3. The method of claim 1 wherein a plurality of first antibodies are used, each having specificity to a different target antigen in the sample.

4. The method of claim 3 wherein said first antibody is a monoclonal antibody immobilized on a substrate.

5. The method of claim 3 wherein the second antibody is detected in step (e) immunologically by an antibody molecule binding to the second antibody.

6. The method of claim 5 carried out in a microfluidic device.

7. The method of claim 5 wherein the first polynucleic acid strand and the second polynucleic acid strand are amine terminated for linkage to an antibody molecule.

8. The method of claim 7 wherein oligonucleotide strands and antibodies comprise a linkage having N-hydroxysuccinimide ester.

9. A method for detecting a target antigen in a sample, using a first antibody and a second antibody, wherein the first antibody and the second antibody can both bind to the target antigen for detection of the target antigen, comprising:
(a) providing said first antibody linked to a first polynucleic acid strand and a second antibody linked to a second polynucleic acid strand,
(b) the first polynucleic acid strand and the second polynucleic acid strand comprising complementary portions that permit hybridization of the first polynucleic acid strand to the second polynucleic acid strand when the first antibody is in proximity to the second antibody by binding to an antigen;
(c) forming, in the sample, a complex wherein a target antigen is bound to said first antibody and also bound to said second antibody, and wherein said first antibody and said second antibody are both monoclonal antibodies to different epitopes on the same target antigen;
(d) incubating the complex under conditions which allow the polynucleic acid strands of step (b) to hybridize each other;
(e) subjecting the complex to a washing step which removes any nonspecifically bound antibody to the target antigen but does not remove any specifically bound antibody, whereby said hybridization occurs; and then
(f) detecting specifically bound antibody by detecting a presence in the sample of an antibody present after a washing step in step (e).

10. A kit for carrying out a method for detecting a number of different target antigens in a sample, comprising:
(a) a plurality of antibody pairs, wherein each pair comprises a first antibody and a second antibody that can concurrently bind to the target antigen, and further wherein
(b) the first antibody is linked to a first polynucleic acid strand and a second antibody is linked to a second polynucleic acid strand, having the first polynucleic acid strand and the second polynucleic acid strand comprise complementary portions that permit specific hybridization of the first polynucleic acid strand to the second polynucleic acid strand when the first antibody is in proximity to the second antibody.

11. The kit of claim 10 further comprising a set of pluralities of antibody pairs directed to different antigens in a sample to be tested by the kit.

12. The kit of claim 10 further comprising instructions for use in assaying antigens in a sample by contacting antigens, instructions comprising use of the plurality of antibody pairs, washing a complex of antigen and antibody pairs, and immunologically detecting complexes present after washing and, optionally, a microfluidic device.

13. An immunological method for reducing cross reactions between one or more antibodies in an assay and an antigen to be assayed, using a first antibody and a second antibody, wherein the first antibody and the second antibody both bind to the same antigen, comprising:
(a) providing a first antibody linked to a first polynucleic acid strand and a second antibody linked to a second polynucleic acid strand, wherein the first polynucleic acid strand and the second polynucleic acid strand comprise complementary portions that permit hybridization to each other when the first antibody is in proximity to the second antibody by binding to the antigen;
(b) forming, in the sample, a complex wherein a target antigen may be bound to said first antibody and also bound to said second antibody;
(c) incubating the complex of step (b) under conditions which allow polynucleic acid strands of step (a) to hybridize to form a polynucleotide complex between the first polynucleic acid strand and the second polynucleic acid strand; and
(d) subjecting the complex in step (c) to a wash step which removes the antigen from the antibody complex, whereby polynucleotide hybridization occurs only if the wash step does not remove the antigen;
(e) detecting a presence in the sample of an antibody having a hybridized strand in a complex with another strand of an antibody of step (a), wherein antibody not having a hybridizing strand is removed in step (d).

14. The method of claim 13 wherein the first antibody and the second antibody are both monoclonal antibodies.

15. The method of claim 13 wherein detecting is done immunologically.

16. The method of claim 15 wherein one antibody, but not another antibody, is immobilized.
